(19) United States
(12) Patent Application Publication
(10) Pub. No.: US 2016/0354333 A1
(43) Pub. Date: Dec. 8, 2016

(54) COMPOSITIONS OF POLYUNSATURATED FATTY ACIDS AND METHODS OF USE THEREOF

(71) Applicant: UND Life Sciences, LLC, Federal Way, WA (US)

(72) Inventor: Undurti N. Das, Federal Way, WA (US)

(21) Appl. No.: 15/172,372

(22) Filed: Jun. 3, 2016

Related U.S. Application Data
(60) Provisional application No. 62/230,307, filed on Jun. 3, 2015.

(51) Int. Cl. A61K 31/202; A61K 47/02; A61K 45/06; A61K 38/21; A61K 38/17; A61K 47/10; A61K 38/19

(52) U.S. Cl. CPC A61K 31/202; A61K 47/10; A61K 47/02; A61K 38/191; A61K 38/21; A61K 38/1709; A61K 45/06

(57) ABSTRACT

Pharmaceutical compositions with selective tumoricial activity comprising polyunsaturated fatty acids or a pharmaceutically acceptable salt or derivative thereof, in an aqueous solution or emulsion are provided. Methods associated with preparation and use of such compositions and methods to selectively cause tumoricial and/or anti-angiogenic action in a neoplastic region, such as cancer, are also provided.

### BACKGROUND

1. Technical Field

The present invention is generally directed to novel compositions comprising a polyunsaturated fatty acid and methods for their preparation and use as therapeutic or prophylactic agents, for example for treatment of cancer.

2. Description of the Related Art

The polyunsaturated fatty acids (PUFAs) are fatty acids having at least two carbon-to-carbon double bonds in a hydrophobic hydrocarbon chain which typically includes 4-28 carbon atoms and terminates in a carboxylic acid group. The PUFAs are classified in accordance with a short hand nomenclature which designates the number of carbon atoms present (chain length), the number of double bonds in the chain and the position of double bonds nearest to the terminal methyl group. The notation “a:b” is used to denote the chain length and number of double bonds, and the notation “n-x” is used to describe the position of the double bond nearest to the methyl group. There are 4 independent families of PUFAs, depending on the parent fatty acid form which they are synthesized. They are:

(i) The “n-3” series derived from alpha-linolenic acid (ALA, 18:3, n-3).

(ii) The “n-6” serried derived from linoleic acid (LA, 18:2, n-6).

(iii) The “n-9” series derived from oleic acid (OA, 18:1, n-9).

(iv) The “n-7” series derived from palmitoleic acid (PA, 16:1, n-7).

The parent fatty acids of the n-3 and n-6 series cannot be synthesized by mammals, and hence they are often referred to as “essential fatty acids” (EFAs). Because these compounds are necessary for normal health but cannot be synthesized by the human body, they must be obtained through the diet.

It is believed that both LA and ALA are metabolized by the same set of enzymes. LA is converted to gamma-linolenic acid (GLA, 18:3, n-6) by the action of the enzyme delta-6-desaturase (d-6-d), and GLA is elongated to form di-homo-GLA (DGLA, 20:3, n-6), the precursor of the 1 series of prostaglandins. The reaction catalyzed by d-6-d is the rate limiting step on the metabolism of EFAs. DGLA can also be converted to arachidonic acid (AA, 20:4, n-6) by the action of the enzyme delta-5-desaturase. AA forms the precursor of 2 series of prostaglandins (PGs), thromboxanes (TXs) and the 4 series leukotrienes (LTs). ALA is converted to eicosapentaenoic acid (EPA, 20:5, n-3) by d-6-d and d-5-d. EPA forms the precursor of the 3 series of PGs, TXs and 5 series of leukotrienes. EPA can be converted to docosahexaenoic acid (DHA, 22:6, n-3) and, in turn, DHA could be retroconverted to EPA. Conjugated linoleic acid (CLA; 18:2) is a group of isomers (mainly 9-cis, 11-trans and 10-trans, 12-cis) of linoleic acid. CLA is the product of rumen fermentation and can be found in the milk and muscle of ruminants {(see, e.g., Brodie et al. (1999), J. Nutr. 129: 602-6; Visonneau et al. (1997), Anticancer Res. 17: 969-73)} LA, GLA, DGLA, AA, ALA, EPA and DHA and CLA are all PUFAs, but only LA and ALA are EFAs. But, several actions of EFAs are also brought about by GLA, DGLA, AA, EPA and DHA and hence, are also called as “conditional EFAs” and hence, for all practical purposes the words EFAs and PUFAs are used interchangeably.

It is known in the art that certain PUFAs and/or their metabolites augment free radical generation, have anti-inflammatory activity, cytotoxic properties towards tumor cells and/or provide substrates for the generation of lipid peroxidation products which have an inhibitory action on cell proliferation. Recent studies have also suggested that lipids could play a significant role in the generation of T_{H}17-Treg cells and yet other investigations showed that blocking PD-1, programmed cell death protein 1, also known as PD-1 and CD279 (cluster of differentiation 279), a cell surface receptor that belongs to the immunoglobulin superfamily and expressed on T cells and pro-B cells could be exploited to activate the immune system and treat cancer.

Despite the known activity of certain PUFA's, they have yet to be successfully used in methods for treatment of cancers, such as glioma, at least in part because of the difficulty associated with formulating and delivering PUFA's. Accordingly, there is a need in the art for improved formulations of PUFA's and methods for their use in treatment of various diseases, including cancer. The present invention provides these and other related advantages.

### BRIEF SUMMARY

In brief, embodiments of the present invention provide a pharmaceutical composition which is capable of selective tumoricidal action when administered. In certain embodiments the composition comprises a polyunsaturated fatty acid, such as gamma linolenic acid, or a pharmaceutically acceptable salt or derivative thereof, a stabilizing agent, saline or phosphate buffered saline, and ethanol wherein the concentration of stabilizing agent and ethanol does not interfere with the tumoricidal or anti-angiogenic activity of the polyunsaturated fatty acid and a solution or emulsion is formed. For example, in some embodiments the concentration of ethanol in the composition ranges from 0.001% to 0.01%. In other embodiments, the polyunsaturated fatty acid is in the free acid form.

In other embodiments is provided, a pharmaceutical composition comprising:

a polyunsaturated fatty acid in the free acid form;

saline or phosphate buffered saline; and

from 0.01% to 0.0001% ethanol.

In some embodiments, a method for treatment of cancer is provided, the method comprising identifying a neoplastic region and administering a therapeutically effective amount of the disclosed pharmaceutical composition to the neoplastic region, for example to selectively cause tumoricidal or anti-angiogenic action in the neoplastic region.

In other embodiments a method for preparing the pharmaceutical composition is provided, the method comprising dissolving a polyunsaturated fatty acid in ethanol to make a first mixture and diluting the first mixture in saline or phosphate buffered saline wherein the final concentration of ethanol ranges from 0.001% to 0.01% is provided.

These and other aspects of the invention will be apparent on reference to the following detailed description.

### BRIEF DESCRIPTION OF THE DRAWINGS

FIG. 1 demonstrates the effect of PGE2 on various cytokines and immunocytes and its effect on tumor cell behavior.

FIG. 2A-B shows the effect of PUFA treatments on alloxan and RIN cells.

FIG. 3 is a schematic representation of an implant for infusion/injection of PUFA compositions into a tumor bed.

FIG. 4A-B shows a comparison of the brain of a patient before and after treatment with a GLA composition according to one embodiment.

FIG. 5 is a bar graph showing the stability of GLA solutions comprising lithium.

FIG. 6 provides stability data for GLA solutions comprising 0.01% ethanol/saline/PBS and lithium or saline/PBS and lithium.

FIG. 7 is additional stability data for GLA solutions comprising 0.01% ethanol/saline/PBS or saline/PBS without added lithium.

FIG. 8 is another bar graph showing cytotoxicity data in cancer cells for GLA solutions in varying concentrations of ethanol.

FIG. 9 provides cytotoxicity data in normal cells for GLA solutions in varying concentrations of ethanol.

### DETAILED DESCRIPTION

In the following description, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless defined otherwise, all technical and scientific terms used herein have the same meaning as is commonly understood by one of skill in the art to which this invention belongs. As used in the specification and claims, the singular form “a”, “an” and “the” include plural references unless the context clearly dictates otherwise.

“Neoplastic”, “tumor”, “anaplasia” and/or “cancer” refers to characterized by abnormal tissue that shows partial or complete lack of structural organisation and functional coordination with normal tissue, and usually forms a distinct mass which may be either benign or malignant. These three terms: “neoplastic”, “tumor” and/or “cancer” are often be used interchangeably herein. As used herein, a “neoplastic region”, “tumor tissue or mass” and/or “cancer mass or tissue” means essentially contiguous region of tissue containing neoplastic tissue. A neoplastic region, tumor and/or cancer region or mass is the smallest volume of tissue that includes the contiguous neoplastic tissue, but may also include normal tissue. Contiguous neoplastic tissues are neoplastic, cancer or tumor tissues separated by distances of less than one centimeter, and do not include distant metastases (which define separate neoplastic, tumor or cancer regions). Although not all neoplastic regions are tumors, the terms “neoplastic region”, “tumor” and “cancer” will often be used interchangeably herein, and the term tumor angiogenesis should be understood to include any blood vessel(s) feeding any type of neoplastic region.

“Polyunsaturated fatty acid” or “PUFA” refers to any acid derived from fats by hydrolysis, or any long-chain (at least 12 carbons) organic acid, having at least two carbon-to-carbon double bonds. Examples of PUFAs include but are not limited to linoleic acid, linolenic acid and arachidonic acid. Even though in some embodiments, the invention specifically deals with PUFAs, it may be mentioned here that butyric acid, a short-chain fatty acid, was also found to have selective cytotoxic action on glioma cells both in vitro and in vivo {Williams et al (2003) Proc Nutrition Soc 62: 107-115}. Hence, in the present definition of EFAs or PUFAs, the name of the short-chain fatty acid butyric acid (BA, 4:0) is included. Thus, the definition of EFAs/PUFAs is extended to those lipids that have anti-cancer actions. Specifically, butyric acid is included. The other short-chain fatty acids such as formic acid, acetic acid, propionic acid, isobutyric acid, valeric acid and isovaleric acid are not included in this definition of “lipids that have anti-cancer actions.” Thus, as used herein, the definition of EFAs/PUFAs is extended to include not only LA, GLA, DGLA, AA, ALA, EPA and DHA but also BA.

“PUFA salt” refers to an ionic association, in solid or in solution, of a anionic form of a PUFA with a cation of a small organic group (e.g., ammonium) or a small inorganic group (e.g., an alkali metal). Salts include those between a PUFA and an alkali metal (e.g., lithium, sodium, potassium), and alkali earth metal (e.g., magnesium, calcium) or a multivalent transition metal (e.g., manganese, iron, copper, aluminum, zinc, chromium, cobalt, nickel).

As used herein, the term “radical” or “free radical” is an atom, molecule, or ion that has unpaired valence electrons. With some exceptions, these unpaired electrons make free radicals highly chemically reactive towards other substances, or even towards themselves: their molecules will often spontaneously dimerize or polymerize if they come in contact with each other. These free radicals are reasonably stable only at very low concentrations in inert media or in a vacuum. A notable example of a free radical is the hydroxyl radical (HO.), a molecule that has one unpaired electron on the oxygen atom. Two other examples are triplet oxygen and triplet carbine (:CH2) which have two unpaired electrons. Though by definition hydroxyl anion (HO—) is not a radical, since the unpaired electron is resolved by the addition of an electron; singlet oxygen and singlet carbene are not radicals as the two electrons are paired. But, for all practical purposes and for the sake of the present invention HO— is considered as a free radical. It is also important to note that free radicals may be created in a number of ways, including synthesis with very dilute or rarefied reagents, reactions at very low temperatures, or breakup of larger molecules. The latter can be affected by any process that puts enough energy into the parent molecule, such as ionizing radiation, heat, electrical discharges, electrolysis, and chemical reactions. Indeed, radicals are intermediate stages in many chemical reactions. Free radicals play an important role in biochemistry, and many other chemical processes. In living organisms, the free radicals superoxide and nitric oxide and their reaction products regulate many processes, such as control of vascular tone, cell mitosis, cell migration and also play a key role in the intermediary metabolism of various biological compounds. Free radicals can serve as messengers referred to as redox signaling. While not wishing to be bound by theory, it is believed that PUFAs including GLA may serve as inducers of free radicals such as superoxide anion, hydroxyl radical, nitric oxide (NO) and other free radicals. Under some well defined conditions, PUFAs including GLA may themselves function as radicals or form radicals and this can be dubbed as PUFA. and GLA. radicals. These PUFA. and GLA. radicals can ultimately produce apoptosis (genetically programmed cell death by inducing damage to DNA) of cancer cells. But will not produce apoptosis of normal cells since normal cells have relevant anti-oxidant defences. Similar to other radicals such as superoxide and OH, both PUFA. and GLA. radicals can also be trapped within a solvent cage or be otherwise bound.

“Anaplasia” refers to a condition whereby cells lose the morphological characteristics of mature cells and their orientation with respect to each other and to endothelial cells. The term also refers to a group of morphological changes in a cell (e.g., nuclear pleomorphism, altered nuclear:cytoplasmic ratio, presence of nucleoli, high proliferation index) that point to a possible malignant transformation. Loss of structural differentiation is seen in most, but not all, malignant neoplasms. The term also includes an increased capacity for multiplication. A lack of differentiation is considered a hallmark of aggressive malignancies. The term anaplasia is used herein to imply dedifferentiation, or loss of structural and functional differentiation of normal cells. It is now known, however, that at least some cancers arise from stem cells in tissues; in these tumors failure of differentiation, rather than dedifferentiation of specialized cells, account for undifferentiated tumors. Anaplastic cells display marked pleomorphism. The nuclei are characteristically extremely hyperchromatic (darkly stained) and large. The nuclear-cytoplasmic ratio may approach 1:1 instead of the normal 1:4 or 1:6. Giant cells that are considerably larger than their neighbors may be formed and possess either one enormous nucleus or several nuclei (syncytia). Anaplastic nuclei are variable and bizarre in size and shape. The chromatin is coarse and clumped, and nucleoli may be of astounding size. More important, mitoses are often numerous and distinctly atypical; anarchic multiple spindles may be seen and sometimes appear as tripolar or quadripolar forms. Also, anaplastic cells usually fail to develop recognizable patterns of orientation to one another (i.e. they lose normal polarity). They may grow in sheets, with total loss of communal structures, such as gland formation or stratified squamous architecture. Anaplasia is the most extreme disturbance in cell growth encountered in the spectrum of cellular proliferations and can be considered as one of the characteristics of cancer/tumor cells.

“Intratumoral” refers to any method of invasively or noninvasively injecting any drug or substance into the neoplastic region/tumor/cancer. Invasive methods include direct injection using a needle, catheter, or any other device that has a catheter/needle whose tip could be inserted into the tumor bed. Noninvasive intratumoral injection method could include injecting into the artery that is “proximal” with respect to a neoplastic region and a site of intra-arterial injection. Noninvasive methods of intratumoral injection may also include injecting into a peripheral vein as a result of which the said drug could reach the tumor mass namely glioma. In both instances of intra-arterial and intravenous methods of injection, if the composition is able to reach glioma in sufficient amounts is considered as noninvasive method of delivery of the drug to the tumor. Thus, for the purposes of definition, it is suggested that direct intratumoral injection of a composition into the tumor bed of glioma is considered as invasive method of delivery and when a composition is able to reach a tumor bed in sufficient amounts even when it is injected into a proximal artery or vein (intra-arterial or intravenous respectively) is considered as an example of noninvasive method of delivery. Thus, when a composition injected direct into the glioma tumor bed by piercing the skull/scalp it is considered as an invasive procedure, and when a composition reaches a glioma tumor bed when it is injected into the artery or vein without piercing the skull or scalp is considered as a noninvasive method of delivery of the invention.

“Neoangiogenesis” refers to formation of new blood vessels. The terms “angiogenesis” and “neoangiogenesis” are used interchangeable to imply that new blood vessels are being formed to supply nutrients to a growing tumor. Angiogenesis is the physiological process through which new blood vessels form from pre-existing vessels. This is distinct from vasculogenesis, which is the de novo formation of endothelial cells from mesoderm cell precursors. The first vessels in the developing embryo form through vasculogenesis, after which angiogenesis is responsible for most, if not all, blood vessel growth during development and in disease. Angiogenesis is a normal and vital process in growth and development, as well as in wound healing and in the formation of granulation tissue. However, it is also a fundamental step in the transition of tumors from a benign state to a malignant one, leading to the use of angiogenesis inhibitors in the treatment of cancer.

As noted above, certain embodiments are directed to compositions of PUFAs, such as GLA, and their use for treatment of cancer, including glioma. Without wishing to be bound by theory, it is believed that the selective tumoricidal action of various PUFAs and, in particular, to GLA can be attributed to one or more than one of the following events: (i) GLA is the most potent PUFA to show differential tumoricidal action; (ii) GLA inhibits PGE2 synthesis, a compound that has pro-inflammatory action, which enhances tumor cell growth and tumor cancer stem cell proliferation; (iii) GLA inhibits angiogenesis that drives and essential for tumor cell growth; (iv) GLA that is instilled into the tumor bed is ingested by tumor infiltrating macrophages, which in turn, converts GLA to hydroxy GLA and/or GLA. radical that is capable of killing tumor cells; (v) GLA inhibits the production of IL-17 that is capable of inducing resistance to anti-VEGF therapies and thus, indirectly enhance tumor cell proliferation; (vi) GLA enhances the action of cytotoxic T cells; (vii) GLA blocks the action of programmed cell death protein (PD-1) and CTLA4 so that immune response against tumor cells is enhanced; (viii) GLA has anti-inflammatory actions such that the inflammatory process involved in the progression of cancer cell growth is inhibited; (ix) GLA has anti-VEGF action and thus, decreases angiogenic processes to tumors that are needed for the cancer to grow; (x) GLA and other PUFAs can form toxic lipid peroxides in tumor cells that could lead to the apoptosis of tumor cells and the formation of these lipid peroxides are more in tumor cells compared to normal cells and thus, GLA is differentially toxic to tumor cells unlike other PUFAs such as AA, EPA and DHA; (xi) studies performed in support of the present invention revealed that GLA enhances the formation of LXA4 that have inhibitory action on the growth of tumor cells and LXA4 also protects normal cells from toxic lipid peroxides; (xii) GLA forms 15-, 12- and 8-OH 20 carbon and 13-OH 18 carbon fatty acid derivatives which are conjugated dienes and/or hydroperoxyl groups of GLA peroxides that are toxic to tumor cells; (xiii) AA, EPA and DHA also form lipid peroxides in the tumor cells but form much less LXA4 in normal cells and hence, normal cells are not protected from the cytotoxic action of lipid peroxides that may explain the more specific and differential toxicity of GLA to tumor cells in comparison to AA, EPA and DHA; (xiv) GLA increases the expression of p53, which can induce apoptosis of tumor cells; (xv) GLA decreases the expression of Bcl-2, an anti-apoptotic gene; and (xvi) GLA by virtue of its ability to form GLA. is capable of enhancing the anti-cancer action of radiation and other chemotherapeutic drugs and thus, is capable of augmenting the anti-cancer action of radiation and conventional anti-cancer drugs. In addition, GLA and other PUFAs produced a significant down-regulation of cytoskeleton-associated genes, in particular three GTPases (RAC1, RHOA, CDC42), three kinases (ROCK1, PAK2, LIMK), two Wiskott-Aldrich syndrome proteins (WASL, WASF2) as well as actin related protein 2/3 complex (ARPC2, ARPC3), cofilin (CFL1) and F-actin content in the tumor cells such that they were unable to undergo mitosis and inhibited their mobility {Schmidt et al (2015) Lipids Health Dis 14:4}.

Moreover, in an extended study, the differences in the tumoricidal action of GLA with regard to other PUFAs, such as DGLA and AA, was shown to be in the way these fatty acids are handled by the tumor cells and the formation of lipid peroxidation products formed from these fatty acids. For instance, GLA is metabolized in the tumor cells to form 15-, 12- and 8-OH 20 carbon and 13-OH 18 carbon fatty acid derivatives that seem to be responsible for the apoptosis of tumor cells. In contrast to this, DGLA could go through an exclusive C-8 oxygenation pathway during COX-catalyzed lipid peroxidation in addition to a C-15 oxygenation pathway shared by both DGLA and AA, and that the exclusive C-8 oxygenation could lead to the production of distinct DGLA's free radical derivatives that may be correlated with DGLA's anti-proliferation activity. As shown below DGLA can go through a unique C-8 oxygenation reaction pathway, in addition to a C-15 oxygenation pathway shared by both DGLA and AA.

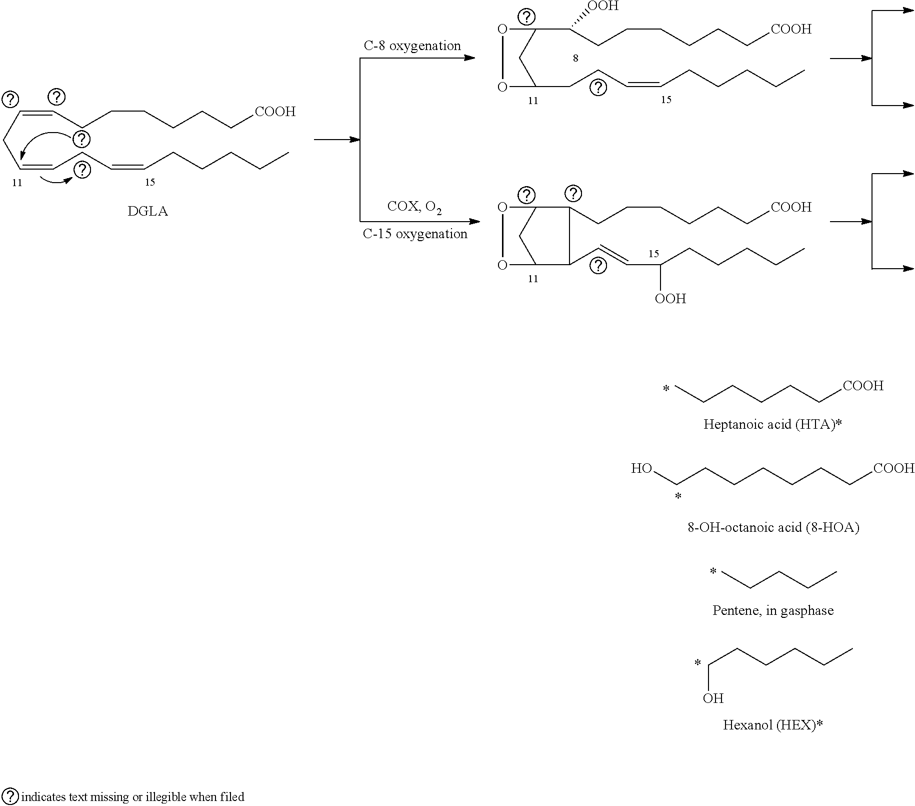

The C-8 oxygenation results in the formation of two exclusive DGLA-derived free radical metabolites, .C₇H₁₃O₂ and .C₈H₁₅O₃, while the C15 pathway produces two common radical metabolites, .C₆H₁₃O and .C₅H₁₁. On the other hand,

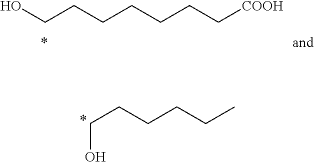

called as 8-OH-octanoic acid (8-HOA) and 1-heaxanol (HEX) respectively are expected to be formed from free radicals by abstracting an H. from the environment in the absence of the spin trapping agent. In a similar fashion, the carbon-centered radicals formed from COX-catalyzed AA peroxidation in vitro were showing an ESR (electron spin resonance)-active peaks and MS (mass spectra) ions of m/z 296, 448, and 548, all stemming from PGF2-type alkoxyl radicals. One of these was a novel radical centered on the carbon-carbon double bond nearest the PGF ring, caused by an unusual β-scission of PGF2-type alkoxyl radicals.

The complementary non-radical product was 1-hexanol (

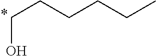

similar to the one that is formed from DGLA as described above) product, instead of the more common aldehyde. Thus, it is evident that the peroxidation products formed from GLA, DGLA and AA are totally different, and this may account for the differences in their tumoricidal action wherein GLA>DGA>AA with regard to their ability to induce apoptosis of tumor cells. Thus, it is evident that the lipid peroxidation end products formed from GLA, DGLA and AA are different and so also the ability of these fatty acids to induce apoptosis of tumor cells explaining why GLA is more potent than DGLA and AA in killing the tumor cells.

Thus, in summary, the formation of lipid peroxidation products and their relevant radical products formed from GLA, DGLA and AA are as follows:

| GLA | DGLA | AA |
|---|---|---|
| 15-OH—C₁₂ FA• | •C₅H₁₁ | •C₅H₁₁ |
| 12-OH—C₂₀ FA• | •C₆H₁₃O | •C₆H₁₃O |
| 8-OH—C₂₀ FA• | •C₇H₁₃O₂ | •C₁₄H₂₁O₄ |
| 13-OH—C₁₈ FA• | •C₈H₁₅O₃ | •C₂₀H₃₄O₅ |

It can be seen that the radicals formed from DGLA and AA as a result of lipid peroxidation process are very similar except for the last two products (namely .C₇H₁₃O₂ and .C₈H₁₅O₃ from DGLA and .C₁₄H₂₁O₄ and .C₂₀H₃₄O₅ from AA) whereas the products formed from GLA seem to be entirely different as shown above. It is evident from these studies that both DGLA and AA when metabolized in tumor cells form similar lipid peroxides (such as .C₅H₁₁ and .C₆H₁₃O) and slightly different lipid peroxides (from DGLA: .C₇H₁₃O₂ and .C₈H₁₅O₃ and from AA: .C₁₄H₂₁O₄ and .C₂₀H₃₄O₅). The formation of at least 2 similar lipid peroxides from DGLA and AA can be ascribed to their common C20 chain length as both DGLA and AA that are C20 molecules, whereas the two different lipid peroxides formed from DGLA and AA (from DGLA: .C₇H₁₃O₂ and .C₈H₁₅O₃ and from AA: .C₁₄H₂₁O₄ and .C₂₀H₃₄O₅) can be ascribed to the differences in the number of double bonds (DGLA has 3 double bonds while AA has 4 double bonds). On the other hand, GLA is an 18 carbon chain length containing 3 double bonds) that may explain the formation of entirely different lipid peroxides compared to those formed from DGLA and AA.

Without wishing to be bound by theory, it is believed that GLA is toxic to glioma cells (and other tumor cells) but not to normal neuronal cells (normal brain cells) because of the absence (or near absence) of GLA in normal brain cells (neuronal cells) while AA, EPA and DHA (DHA>AA>EPA) are present in abundant amounts in them (human brain is known to be rich in AA, EPA and DHA) {Svennerholm et al (1968) J Lipid Res 9: 570-579; Martinez et al (1998) J Neurochem 71: 2528-2533}. Thus, glioma cells may be more sensitive to the toxic actions of GLA but not to that of AA, EPA and DHA. On the other hand, normal neuronal cells of the brain contain not only large amounts of AA, EPA and DHA but may also have ability to convert GLA to AA due to the action of delta-5-desaturase so that AA formed is merged with the cell content of AA such that it is no longer toxic to normal neuronal cells. In contrast, glioma cells (and cancer cells) do not have this capacity to convert GLA to AA due to the low activity or almost absence of delta-5-desaturase in tumor cells and so GLA will remain in free form that leads to its conversion to GLA-derived lipid peroxides that are toxic to tumor cells. This may explain as to why GLA is able to induce selective apoptosis of glioma cells while being non-toxic to normal neuronal cells.

Further, Prostanoids such as PGD2, PGE2, PGF2a, PGI2 and thromboxane A2, produced from AA in response to various stimuli by the action of cyclooxygenase and respective synthases are present in significant amounts at the sites of inflammation (Hilkens et al (1995) Eur J Immunol 25: 59-63; Yao et al (2009) Nat Med 15: 633-640}. Of all, PGE2 has immunomodulatory action and suppresses TH1 differentiation. Stimulatory action of PGE2 on T_{H}17 differentiation or expansion in vitro is noted. PGE2 also has a role in the impairment of CTL function in co-ordination with PD-1. PGE2 has pro-inflammatory actions and the immunosuppressive function of PGE2 may be responsible for the immunosuppression seen in cancer and its ability to limit the functions of NK cells, CD4 and CTLs {Linnemeyer et al (1993) J Immunol 150: 3747-3754; Sreeramkumar et al (2012) Immunol Cell Biol 90: 579-586}. The increase in the production of PGE2 both by the tumor cells and monocytes/macrophages infiltrating the tumor has been held responsible for the defective cellular immune response;

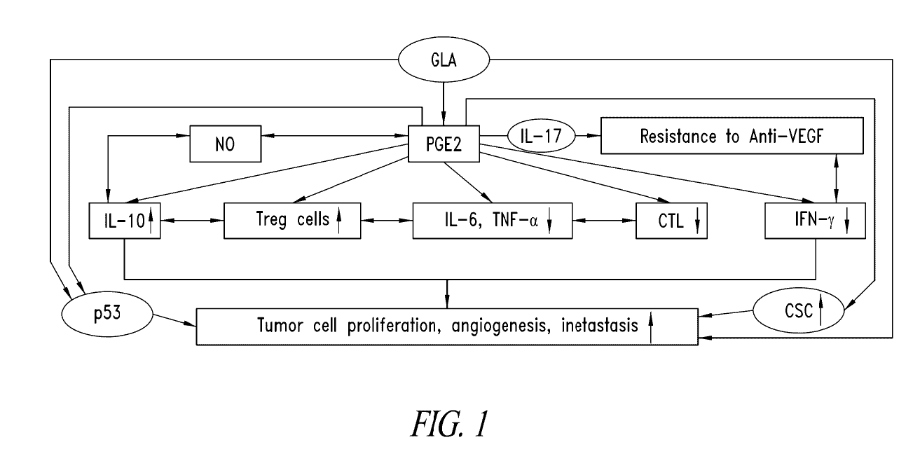

hypercalcemia, tumor cancer cell proliferation, tumor angiogenesis and resistance of tumor to anti-VEGF therapy (see FIG. 1). In addition, PGE2 may have the ability to modulate NO generation. In contrast to the previous results, we also noted that both COX and LOX inhibitors (indomethacin and nordihydroguaiaretic acid when used as 60 and 20 mg/ml respectively) enhanced the growth of IMR-32 cells. Though this growth promoting action of indomethacin and NDGA could be attributed to their non-specific anti-oxidant actions, it is equally possible that some unknown products have been generated from PUFAs that have growth promoting actions. In order to verify this possibility, we also studied the effect of LXA4, resolvins and protectins formed from AA, EPA and DHA and noted that these anti-oxidant metabolites inhibited the growth of IMR-32 cells (Sailaja et al (2014) PLOS One 9: e114766}. Thus, PGE1, PGE2, LTD4, LXA4, resolvins and protectins seem to possess growth inhibitory action on IMR-32 cells. These results imply that the balance between various eicosanoids formed from their precursors and the cellular content of PUFAs in the surrounding milieu of tumor cells (that is normal cells surrounding the tumor cells) may determine whether tumor cells are induced to proliferate or suppressed from further growth. Accordingly, treatment of tumor cells with PUFAs according to embodiments of the invention is believed to be a viable treatment method for cancers, such as glioma.

There are several advantages of PUFA/GLA treatment as described in certain embodiments of the invention. In some embodiments, a single injection per day for 7 to 10 days at separate times is adequate to produce almost permanent regression of the tumor, suppression of the tumor feeding vessels, prevent angiogenesis/neoangiogenesis with no or very little recurrence of the tumor. PUFAs/GLA and their salts are non-antigenic, are known to be relatively safe and stable in the dosages described herein.

Without wishing to be bound by theory, certain aspects of the present invention are believed to relate, at least in part, to the discovery of the novel and highly beneficial action of PUFAs, such as GLA, to induce selective killing and elimination of glioma tumor cells, regress tumor mass and/or prevent it from further growth without any significant side effects. In some embodiments, this effect is particularly observed when the PUFA, such as GLA, is administered directly to the tumor bed (e.g., injected into the glioma mass). It is believed that the selective tumoricidal action of GLA or other PUFAs administered is due to the generation of toxic free radicals to a significant extent only in the tumor cells but not in the normal cells.

All PUFAs and in particular, GLA are not water soluble as a result of which it is difficult to deliver to tumor cells. Solvents that are used to dissolve PUFAs and, in particular GLA have biological actions that could render GLA inactive or interfere with its beneficial actions especially, tumoricidal action. For example, DMSO (dimethyl sulfoxide) is a solvent that could be used to dissolve various PUFAs including GLA. But, DMSO is a potent anti-oxidant and it interferes with the tumoricidal action of PUFAs and in particular with that of GLA. Similarly, PUFAs and in particular GLA are soluble in other lipid solvents such as ethyl acetate, methanol, chloroform, acetone, hexane, isopropanol, methyl-tert-butyl ether (MTBE), or detergent such as Triton X-114. But, all these solvents themselves have potent cytotoxic actions and thus, PUFAs including GLA dissolved in these solvents show toxic action on normal cells.

In view of this solubility issue and non-availability of a suitable non-toxic solvent to dissolve various PUFAs including GLA for their appropriate delivery to human tissues, especially to the brain, no progress has been made in the parenteral delivery of various PUFAs and especially that of GLA for various human diseases including glioma. Identification and/or development of a suitable solvent for PUFAs and especially for GLA (since in the absence of a suitable solvent GLA cannot be injected into human brain parenchyma) is needed so that its tumoricidal action can be exploited in an appropriate fashion for the treatment of neoplasia and more so for glioma. This is so since, if the solvent used to dissolve PUFAs including GLA is toxic then it is likely to produce significant side effects especially when these lipids are injected into the parenchyma of the brain. Hence, a suitable solvent should be identified or developed to dissolve PUFAs including GLA such that the solvent is non-toxic and delivery of PUFAs including GLA can be performed safely. Furthermore, the solvent used should make PUFAs including GLA, at least, partially water soluble so that further dilution of the solution is possible to deliver the required amount(s) of the fatty acid.

In the absence of such a suitable water soluble or at least partially water soluble solvent system for dissolving and delivering PUFAs including GLA, one will not be able to deliver appropriate amounts of lipid (such as GLA and other PUFAs) needed to produce the desired actions. In the absence of such a suitable water soluble or at least partially water soluble system, the amount(s) of PUFAs including GLA delivered to the tissues will be either too high or too low but not appropriate. Thus, development of a suitable solvent system for the delivery of PUFAs including GLA is needed so that even if accidentally PUFAs including GLA is injected into normal tissues including brain no side effects will occur due to the solvent used for delivering PUFAs, especially GLA into the brain parenchyma. This is important since, PUFAs; especially GLA by itself is not toxic to normal cells and to rule out the possibility that the side effects observed are due to the solvent system used for the delivery of PUFAs including GLA.

Without wishing to be bound by theory, it is believed that, in certain embodiments, there is an interaction between the PUFA and the solvent system used to dissolve it which may account for the effectiveness of the treatment. Thus, the newly discovered solvent system (and method for preparation of the composition) used which, in certain embodiments, comprises pure ethyl alcohol and a stabilizing agent, such as lithium, is believed to synergistically interact with the PUFAs to produce a therapeutic effect which is unexpectedly different than the effect of either PUFA or the solvent and/or agent/stabilizing agent alone.

The optional stabilizing agent can be present in various amounts. For example, in some embodiments the optional stabilizing agent is present in the composition in amounts ranging from about 1 picogram/gram of PUFA to about 10 micrograms/gram of PUFA. In some embodiments the optional stabilizing agent is present in the composition in amounts ranging from about 1 picogram/gram of PUFA to about 100 nanograms/gram of PUFA. In other embodiments, the concentration of stabilizing agent in the composition ranges from about 1 picogram/gram of PUFA to about 10 nanograms/gram of PUFA. In other embodiments, the concentration of stabilizing agent in the composition ranges from about 1 picogram/gram of PUFA to about 1 nanograms/gram of PUFA. In other embodiments, the concentration of stabilizing agent in the composition ranges from about 1 picogram/gram of PUFA to about 100 picograms/gram of PUFA. In other embodiments, the concentration of stabilizing agent in the composition ranges from about 1 picogram/gram of PUFA to about 10 picograms/gram of PUFA.

Accordingly, in some embodiments the present invention provides a composition comprising a PUFA, such as GLA, ethyl alcohol and an optional stabilizing agent. The present inventor unexpectedly discovered that when the ethyl alcohol concentration is outside the optimal range in any solution wherein GLA/PUFA is present, the tumoricidal action of GLA/PUFA is suboptimal and the PUFA unstable. This is an unexpected observation since it is not typically expected that any solvent used for dissolving an active chemical would be so critical for the stability and activity of the active chemical. However, in the case of PUFAs, including GLA, this was found to be true.

Thus, as already discussed above, the surprising and novel observation of the inventor is the fact that any deviation in the solvent content outside the optimal range in a given solution in which GLA is present, the activity, stability and its tumoricidal action were altered such that GLA became relatively inactive, unstable and its tumoricidal action inefficient. In general, it is believed that two or more molecules or drugs or compounds that have similar properties (with similar or different mechanisms of action(s)) will enhance the final action of each other. For instance, in cancer therapy two or more drugs with different mechanism(s) of action are employed so that tumor cells are killed to give relief to the patient. Thus, use of more than two or three drugs in different combinations is often used in cancer chemotherapy. Based on similar principle, it has been proposed that a combination of anti-angiogenic molecules: endostatin and angiostatin when combined with salts of PUFAs will potentiate each other's action especially when given intra-arterially and occlude tumor feeding vessels (see U.S. Pat. No. 6,380,253). Though this argument looks reasonable, to the surprise of the inventor it was found that this is not the case.

The inventor unexpectedly discovered that the free acid form of PUFA/GLA is more potent than sodium salt, ethyl ester, methyl ester or other forms of salts, esters, glycerides, amides, or phospholipids, or alkylated, alkoxylated, halogenated, sulfonated, or phosphorylated forms of the fatty acid. In general, it was believed that the potency of actions of a free acid form is equal to or similar to sodium salt, ethyl ester, methyl ester or other forms of salts, esters, glycerides, amides, or phospholipids, or alkylated, alkoxylated, halogenated, sulfonated, or phosphorylated forms of the fatty acid. However, it was unexpectedly discovered that the free acid of PUFA/GLA is the most potent in bringing about its anti-cancer action in comparison to sodium salt, ethyl and methyl esters and other forms of fatty acid.

The present inventor also unexpectedly discovered that the anti-cancer actions of interferon (IFN) and tumor necrosis factor-a (TNF-a) need the presence of substantial amounts of cellular content of GLA, AA, EPA and DHA (˜GLA≧AA>EPA>DHA). For instance, when IFN-sensitive tumor cells were exposed to IFN, 24-48 hours after the addition of IFN accumulation of lipid droplets in the cytoplasm surrounding the nucleus was seen that coincided with the apoptosis of the tumor cells. On the other hand, when IFN-resistant cells were exposed to IFN (even very high doses of IFN), there was a significantly less amount of apoptosis of cancer cells and no accumulation of lipid droplets were seen in these cells. In contrast to these results, when IFN-resistant cells were pre-treated with GLA/AA and then exposed to IFN both induction of apoptosis and accumulation of lipid droplets in the tumor cells were seen.

Further studies have also revealed that PUFAs such as GLA, AA, EPA and DHA can prevent or ameliorate the side effects of anti-cancer agents such as gamma-radiation and cis-platinum to the bone marrow cells of mice. Thus, it appears that when PUFAs and conventional anti-cancer drugs/agents are given together they not only act to potentiate the cytotoxic action of each other on the tumor cells and thus, produce synergistic and/or additive action on their ability to eliminate the tumor cells but it will also lead to elimination, reduction, or amelioration of the side effects of conventional anti-cancer agents. Since, PUFAs are able to potentiate the cytotoxic action(s) of conventional anti-cancer drugs (agents) and cytokines; it is also possible that this will lead to a significant reduction in the doses of these latter agents without compromising the ultimate benefit namely, elimination of tumor cells or the tumor. Accordingly, certain embodiments are directed to methods comprising administering a PUFA (e.g., GLA) containing compositions as described herein and a conventional anti-cancer drug to a subject in need thereof.

Accordingly, in one embodiment is provided a pharmaceutical composition comprising:

a polyunsaturated fatty acid in the free acid form;

saline or phosphate buffered saline; and

from 0.01% to 0.0001% ethanol.

In some embodiments, the polyunsaturated fat is linoleic acid, gamma-linolenic acid, di-homo gamma linolenic acid, arachidonic acid, alpha-linolenic acid, eicosapentaenoic acid, docosahexaenoic acid, conjugated linoleic acid, or combinations thereof. For example, in certain embodiment the polyunsaturated fatty acid is gamma-linolenic acid.

In various different embodiments, the polyunsaturated fatty acid is conjugated to a tumor necrosis factor a, an interferon, an anti-neoplastic agent, an antibody, an anti-cancer agent or combinations thereof. For example, in some embodiments the anti-cancer agent is vincristine, adriamycin, doxorubicin, cyclophosphamide, cis-platinum, L-asparaginase, procarbazine, camptothecin, taxol, 5-fluorouracil, busulfan, or combinations thereof.

In other different embodiments the pharmaceutical composition further comprises a tumor necrosis factor a, an interferon, an anti-neoplastic agent, an antibody, an anti-cancer agent or combinations thereof. For Example, in some embodiments the anti-cancer agent is vincristine, adriamycin, doxorubicin, cyclophosphamide, cis-platinum, L-asparaginase, procarbazine, camptothecin, taxol, 5-fluorouracil, busulfan, or combinations thereof

In some embodiments, the molar ratio of polyunsaturated fatty acid to tumor necrosis factor a or interferon ranges from 2:1 to 1:3.

In other embodiments, the concentration of polyunsaturated fatty acid in the pharmaceutical compositions ranges from 5 to 75%, or from 25 to 75%.

The present inventor has unexpectedly found that certain stabilizing agents, such as lithium, increase the stability of solutions comprising PUFA, such as GLA. Accordingly, in some embodiments the pharmaceutical compositions further comprises a stabilizing agent, such as lithium.

Some embodiments also provide methods of selectively causing anti-angiogenic action in a neoplastic region, such as a tumor region, with the result that new blood vessels and collaterals are not formed to sustain the neoplasia.

It is known that malignant tumors are angiogenesis-dependent diseases. But, tumor-associated angiogenesis is a complex, multi-step process which can be controlled by both positive and negative factors. It appears that angiogenesis is necessary, but not sufficient, as the single event for tumor growth {Chen et al (1999) Cancer Res 59: 3308-3312}. But, it is evident from several studies that angiogenesis may be a common pathway for tumor growth and progression. Though several anti-angiogenic agents are being tried to arrest tumor growth, these are not without problems. Since the majority of these agents are proteins/peptides, their long-term use may lead to the development of antibodies which can neutralize their action. These anti-angiogenic substances need to be given repeatedly and some of them are unstable and are difficult to produce in large amounts. But, it needs to be understood that for the production of angiogenic factors, which are produced mainly by the tumor cells {Meister et al (1999) Eur J Cancer 35: 445-449}; macrophages {Xiong et al (1998) Am J Pathol 153: 587-598}; by leukocytes and platelets {Salven et al (1999) Clin Cancer Res 5: 487-491}; and by endothelial cells (Namiki et al (1995) J Biol Chem 270: 31189-31195}a viable tumor mass or tumor cells is needed, which is the stimulus for the production of angiogenic factors. In other words, if the tumor cells are killed then they (tumor cells) will not be able to produce angiogenic factors by themselves and incite the macrophages, endothelial cells, leukocytes and platelets to produce VEGF and other angiogenic factors for the tumor cells to grow and tumor to progress. As a result of this, it will ultimately lead to elimination of the tumor mass itself. Thus, the most important strategy to eliminate tumor mass is to kill tumor cells directly with little or no action on surrounding normal cells.

In this context, embodiments of the present invention provides for use of the disclosed compositions in methods for selectively reducing (i) the growth and inducing apoptosis of the tumor cells; (ii) inhibiting the production of angiogenic factors including VEGF; (iii) blocking PGE2 production; (iv) preventing angiogenesis; (v) rendering macrophages infiltrating the tumor bed to produce more hydroxy fatty acids that are toxic to tumor cells; (vi), decreasing IL-17 synthesis; (vii) enhancing the action of cytotoxic T cells and decreasing the expression of PD-1; (viii) suppressing inflammation locally; (ix) enhancing the expression of p53; (x) inhibiting the expression of Bcl-2; and (xi) increasing the production of LXA4 in the surrounding normal cells when GLA is injected in to at least a portion of the neoplastic region, preferably, glioma (especially, glioblastoma multiforme and other types of primary brain tumors and secondaries arising from other cancers elsewhere in the body that have metastasized to brain). In one embodiment, the invention provides methods in which (a) a portion of the tumor region is identified and (b) a therapeutically effective amount of a solution of GLA is injected, thereby selectively inducing the tumor cells to undergo apoptosis in a period of less than 24 hours. In preferred embodiments, the amount of GLA present in the administered solution is sufficient not only to cause apoptosis of tumor cells but also to decrease the production of angiogenic factor including VEGF by the tumor cells and the surrounding cells (including normal cells) in a period of less than 24 hours.

In certain embodiments, the invention provides a method for treatment of cancer comprising:

identifying a neoplastic region in a subject in need thereof;

administering a therapeutically effective amount of the pharmaceutical composition disclosed herein to the neoplastic region.

In some embodiments, administering comprises administering the pharmaceutical composition intratumorally. In other embodiments, administering comprises administering the pharmaceutical composition via a catheter placed in a tumor bed.

In various different embodiments, the method comprises administering the pharmaceutical composition daily. In some embodiments, the composition is steadily and predictably administered in therapeutically effective amounts over 1-10 days.

In some more specific embodiments,the neoplastic region comprises glioma.

In yet more embodiments of the foregoing method, the pharmaceutical composition comprises gamma-linolenic acid.

In preferred embodiments, the therapeutically effective amount of GLA is between 0.5 mg to 50 gm per day, most preferable between 1.0 mg to 30 mg per day irrespective of the size of the tumor mass.

In some embodiments of the invention, GLA is in the form of a free acid and is made soluble in water using a distinct method of solubilizing it without losing its activity (since normally GLA becomes inactive rapidly when present in water) using a stabilizing agent that not only makes GLA stable but does not interfere with its action(s) and injecting in to the brain tumor mass.

In some embodiments of the invention, in addition to injecting GLA in to the tumor mass, a magnetic resonance image (MM) or computed tomography (CT) scan images of the glioma/secondary tumor in the brain are taken and recorded before and after the injection. Second and subsequent MRI, CT or radiographic images of the glioma region are taken every 24 hours, most preferably 24 hours after 7 days or 10 days of injection or administration of GLA into the tumor mass to assess and record the extent of remission of the glioma.

In some embodiments, the injected fatty acid (GLA) could be a PUFA. This PUFA could be an EFA. In certain preferred embodiments, the EFA is selected from linoleic acid, GLA, DGLA, AA, ALA, EPA, DHA and conjugated linoleic acid (CLA).

In some embodiments, the PUFA is administered in the form of free acid, or a salt, such as lithium salt, a sodium salt, magnesium salt, a manganese salt, an iron salt, a copper salt, or an iodide salt. In some preferred embodiments, the PUFA is in the form of a fatty acid derivative, such as a glyceride, ester, ether, amide, or phospholipid, or an alkylated, alkoxylated, halogenated, sulfonated, or phosphorylated form of the fatty acid.

In some embodiments of the invention, the neoplastic tissue is a tumor. In particular, the neoplastic tissue may be a glioma.

In some embodiments of the invention, in addition to the GLA, a therapeutically effective amount of a compound selected from tumor necrosis factor, anti-cancer drugs, lymphokines, and specific polyclonal or monoclonal antibodies is injected in combination with GLA. In preferred embodiments, the lymphokine is alpha-interferon or gamma-interferon.

In other embodiments, the GLA is covalently conjugated with a pharmaceutical agent chosen from TNF, a-interferon, y-interferon, an antibody, vincristine, adriamycin, doxorubicin, cyclophosphamide, cis-platinum, L-asparaginase, procarbazine, camptothecin, taxol, 5-fluorouracil or busulfan.

In another aspect, the invention provides pharmaceutical compositions of GLA or PUFA, or free acid or salt of PUFA, in combination with an anti-neoplastic agent.

In one aspect, the present invention provides a method of preparation of GLA (free acid form) such that it is made more water soluble, more stable at room temperature and is more active than the methyl, ethyl, or sodium and other types of salts of GLA such that it is able to enter the tumor cell better and bring about its selective tumoricidal action in a desirable fashion. All PUFAs and in particular, GLA are not water soluble as a result of which it is difficult to deliver to tumor cells. This is so since, solvents that are used to dissolve PUFAs and, in particular GLA have biological actions that could render GLA inactive or interfere with its beneficial actions especially, tumoricidal action. For example, DMSO (dimethyl sulfoxide) is a solvent that could be used to dissolve various PUFAs including GLA. But, DMSO is a potent anti-oxidant and it interferes with the tumoricidal action of PUFAs and in particular with that of GLA. Similarly, PUFAs and in particular GLA are soluble in other lipid solvents such as ethyl acetate, methanol, chloroform, acetone, hexane, isopropanol, methyl-tent-butyl ether (MTBE), or detergent such as Triton X-114. But, all these solvents themselves have potent cytotoxic actions and thus, PUFAs including GLA dissolved in these solvents showed toxic action on normal cells. In view of this solubility issue and non-availability of a suitable non-toxic solvent to dissolve various PUFAs including GLA for their appropriate delivery to human tissues, especially to brain, no progress has been made in the parenteral delivery of various PUFAs and especially that of GLA for various human diseases including glioma. Identification and/or development of a suitable solvent for

PUFAs and especially for GLA (since in the absence of a suitable solvent GLA cannot be injected into human brain parenchyma) is an essential so that its tumoricidal action can be exploited in an appropriate fashion for the treatment of neoplasia and more so for glioma. This is so since, if the solvent used to dissolve PUFAs including GLA is toxic then it is likely to produce significant side effects especially when these lipids are injected into the parenchyma of the brain. Hence, it is critical that a suitable solvent is identified or developed to dissolve PUFAs including GLA such that the solvent is non-toxic and delivery of PUFAs including GLA can be performed safely. Furthermore, the solvent used should make PUFAs including GLA, at least, partially water soluble so that further dilution of the solution is possible to deliver the required amount(s) of the fatty acid. In the absence of such a suitable water soluble or at least partially water soluble solvent system for dissolving and delivering PUFAs including GLA, one will not be able to deliver appropriate amounts of lipid (such as GLA and other PUFAs) needed to produce the desired actions. In the absence of such a suitable water soluble or at least partially water soluble system, the amount(s) of PUFAs including GLA delivered to the tissues will be either too high or too low but not appropriate. Thus, development of a suitable solvent system for the delivery of PUFAs including GLA is critical so that even if accidentally PUFAs including GLA is injected into normal tissues including brain no side effects will occur due to the solvent used for delivering PUFAs, especially GLA into the brain parenchyma. This is important since, PUFAs; especially GLA by itself is not toxic to normal cells and to rule out the possibility that the side effects observed are due to the solvent system used for the delivery of PUFAs including GLA. In this context, the inventor noted after several experiments that dissolving GLA and other PUFAs in pure ethyl alcohol initially followed by subsequent dilutions in sterile saline or PBS (phosphate buffered saline, pH 7.4) such that the final concentration of ethyl alcohol is no more than 0.01% to 0.001%. being bound to any particular theory of the invention.

Finally without being bound to any particular theory of invention, it is believed that there is an interaction between the PUFA and the solvent system used to dissolve it which may account for the effectiveness of the treatment. Thus, the solvent system used in embodiments of the present invention, which comprises pure ethyl alcohol, and the stabilizing agent are believed to synergistically interact with the PUFAs to produce a therapeutic effect which is unexpectedly different than the effect of either PUFA or the solvent agent/stabilizing agent alone.

Accordingly, in one embodiment is provided a method for preparing the disclosed pharmaceutical composition, the method comprising,

dissolving a polyunsaturated fatty acid in free acid form in a solution comprising ethanol to form a first mixture; and

diluting the first mixture in saline or phosphate buffered saline such that the final concentration of ethanol ranges from 0.001% to 0.01%.

In some embodiments, the polyunsaturated fatty acid is gamma-linolenic acid.

In other embodiments, the method further comprises including a stabilizing agent, for example lithium, in the first mixture or in the diluted first mixture.

There are several advantages of PUFA/GLA treatment of the invention. As shown below, a single injection per day for 7 to 10 days at separate times is adequate to produce almost permanent regression of the tumor, suppression of the tumor feeding vessels, prevent angiogenesis/neoangiogenesis (formation of new blood vessels) with no or very little recurrence of the tumor. PUFAs/GLA and their salts are non-antigenic, are known to be relatively safe in the dosages employed and are stable as prepared and used.

The invention in one aspect provides methods of inhibiting the growth of tumor cells, blocking of the blood vessels that feed the tumor and prevents development of new blood vessels to feed the tumor (a process called as angiogenesis or neoangiogenesis as defined previously above).

The invention in another aspect provides methods for treating neoplasia, especially glioma and for facilitating visualization of remission of a neoplasia which is responsive to treatment, comprising the steps of (a) locating the neoplastic region in the brain and spinal cord; (b) obtaining an initial radiographic image of the region; (c) injecting into the tumor mass a preparation of PUFA that may consist of a mixture of (i) PUFA/GLA solution dissolved in the most suitable solvent and consisting of the stabilizing agent; (ii) a solution of at least one PUFA chosen from LA, GLA, DGLA, AA, ALA, EPA, DHA, CLA and BA; and (iii) obtaining second and optionally, subsequent radiographic images of the neoplastic region after predetermined lapses of time; and comparing the initial radiographic images with the second and/or subsequent radiographic images to assess the extent of remission of the neoplasia.

The invention in another aspect provides methods of causing necrosis in a neoplastic region (i.e. a cancerous tumor) by inducing apoptosis of tumor cells and/or inhibiting blood supply to the neoplastic region, comprising the steps of (a) locating the neoplastic region; (b) injecting into the tumor mass a mixture of (i) PUFA/GLA solution dissolved in the most suitable solvent and consisting of the stabilizing agent; and (ii) a solution of at least one PUFA chosen from LA, GLA, DGLA, AA, ALA, EPA, DHA, BA, and CLA; (c) waiting for a predetermined time period and assessing a degree of necrosis in the neoplastic region; (d) repeating the treatment if necessary to increase the necrosis.

In yet another aspect, the invention provides methods of treating mammalian cell proliferative disorders using a solution of a PUFA, or a combination of PUFAs, administered intra-tumorally. The methods are as described above with respect to a neoplastic region.

In each of the foregoing embodiments, the PUFA, such as GLA, is preferably in the form of a free acid or any other suitable salt form, and is preferably administered in combination with a stabilizing agent and the final ethyl alcohol concentration is no more than 0.01 to 0.001%. The maintenance of the appropriate concentration of ethyl alcohol and the stabilizing agent (e.g., not more than 0.01 to 0.001%) is believed to contribute to the fact that the GLA/PUFA is stable and the solvent and/or the stabilizing agent do not interfere with the tumoricidal action of the said fatty acid. The inventor also observed to his surprise that when the ethyl alcohol concentration is more than 0.01% and less than 0.001% in any solution wherein GLA/PUFA is present, the action of GLA/PUFA is suboptimal and became unstable and its (GLA/PUFA) tumoricidal action is not optimal. This is an unexpected observation since, it is never expected that any solvent that is used for dissolving an active chemical could be so critical for the stability and activity of the active chemical that has been dissolved in a given solvent. But, in the case of GLA this was found to be true. Any increase in the solvent content above 0.01% and lower than 0.001% in a given solution in which GLA is present, the activity, stability and its tumoricidal action were altered such that GLA became relatively inactive, unstable and its tumoricidal action inefficient.

Thus, as already discussed above, the surprising and novel observation of the inventor is the fact that any increase in the solvent content above 0.01% and lower than 0.001% in a given solution in which GLA is present, the activity, stability and its tumoricidal action were altered such that GLA became relatively inactive, unstable and its tumoricidal action inefficient. This surprising observation that the concentration of ethyl alcohol in the final solution containing GLA/PUFAs and the type of fatty acid (such as free acid form) are the critical factors that need specific and particular attention both for stability of the active compound (GLA in this instance) and to obtain its beneficial action namely tumoricidal action. These properties of the GLA free acid is seen only when the solvent ethyl alcohol content in the final solution is between 0.01% to 0.001% that cannot be anticipated from the prior art. Based on the prior art, those skilled in the art would have anticipated that the injection of GLA/PUFAs into the brain parenchyma would result in (i) sudden death; (ii) convulsions due to the formation of lipid peroxides that are known to be toxic to cells; (iii) cause occlusion of blood vessels in the brain and that would have led to the onset of stroke or paralysis and/or (iv) other deleterious actions. It may also be noted that neuronal cells of the brain are more amenable to lipid peroxidation due to their high content of unsaturated fatty acids. In contrast to these anticipated actions based on the prior art, the inventor noted that GLA/PUFAs when prepared as defined above and in the solvent system as described and administered as outlined, it produced a dramatic beneficial action that is totally unanticipated and against all prediction and produced death of only tumor cells with no action on normal neuronal cells of the brain and regression of tumor/glioma. This differential action of GLA/PUFAs only on tumor cells but not on normal cells including normal neuronal cells is due to the fact that tumor cells are deficient in anti-oxidants. As a result, when lipid peroxides are formed in tumor cells, they are unable to withstand the toxic actions of these lipid peroxides and so undergo apoptosis. In contrast, normal cells including normal neuronal cells have the ability to enhance their anti-oxidant capacity when significant amount of lipid peroxides are formed so that they are able to overcome the apoptotic action of lipid peroxides. Furthermore, normal cells have capacity to degrade excess lipid peroxides formed while tumor cells cannot do so. As a result, lipid peroxides continue to accumulate in tumor cells while this does not happen in normal cells. Thus, tumor cells are not only deficient in PUFAs but also have limited capacity to generate additional anti-oxidants to overcome the toxic actions of lipid peroxides. On the other hand, normal cells including neuronal cells are able to generate additional anti-oxidants to neutralize the toxic actions of lipid peroxides and degrade the lipid peroxides formed.

Although the invention is described primarily as it relates to humans, it is envisaged that the methods of the invention are equally applicable to other mammals, including large domesticated mammals (e.g., race horses, breeding cattle) and other smaller domesticated animals (e.g., house pets, dogs).

The present invention employs PUFAs, preferably in the form of free acid, to selectively eliminate tumor cells. Preferred PUFAs include, but are not limited to GLA, AA, DHA, EPA, DGLA, ALA, LA, CLA and BA. Other preferred PUFAs include derivatives of the aforementioned PUFAs, including glycerides, esters, amides, or phospholipids, or alkylated, alkoxylated, halogenated, sulfonated, or phosphorylated forms of the fatty acid. In most preferred embodiments, the PUFA is GLA, AA or DHA.

The PUFA is preferably administered in the form of a free acid solution. Other suitable forms include in the form of a salt solution. Suitable salt include salts of a PUFA with cation of a small organic group (ammonium) or a small inorganic group (e.g., an alkali metal or alkali earth metal). Preferred referred salts are those between a PUFA and an alkali metal (e.g., lithium, sodium, potassium), an alkali earth metal (e.g., magnesium, calcium) or a multivalent metal (e.g., manganese, iron, copper, aluminium, zinc, chromium, cobalt, nickel). Most preferred are free acids of the said fatty acid. Combinations of free acids or salts may also be employed.

When the PUFAs or PUFA free acids are administered in a suitable solvent as discussed above, the solution may be formed into an emulsion.

In one aspect, the invention provides pharmaceutical compositions comprising a PUFA, or a PUFA salt, a PUFA acid, and TNF-a, IFN and a known anti-cancer drug chosen from the group consisting of vincristine, adriamycin, doxorubicin, cyclophosphamide, cis-platinum, 5-fluorouracil, L-asparaginase, procarbazine, camptothecin, taxol and busulfan in a solution, or in an emulsion. The PUFA and TNF-α/IFN and anti-cancer drug may be separate chemical moieties combined in a solution or emulsion, or they may be covalently conjugated. The TNF-α/IFN agents may be mixed with the PUFA solution described above, either to form a new solution or to form an emulsion, or they may be chemically conjugated to the PUFAs of the invention via standard chemistries. Preferably the PUFA solution is mixed with such a TNF-α/IFN in a ratio of at least about 2:1, or about 1:1 or about 1:1.5, or about 1:2 or about 1:3 (volume/volume or Mol: Mol). Most preferably the ratio is between 1:1.5 and 1:3 (volume/volume). The PUFA solution may be safely administered to a typical patient with glioma or any other tumor in an amount of about 1 mg to 500 mg in a volume of about 0.5 mL to 10 mL/m², but the attending physician should consider all relevant medical factors in determining the appropriate dosage for any specific patient. The preferred PUFA solution and ratios of such a product (a combination of a PUFA and TNF-α/IFN) are as disclosed above. Preferably the final concentration of the PUFA in such a product is at least 5%, preferably at least 25%, and most preferably about 25-75%.

In another aspect, the invention provides pharmaceutical compositions comprising a PUFA, or a PUFA salt, and an anti-cancer agent in solution, or in an emulsion. The PUFA and anti-cancer agent may be separate chemical moieties combined in the solution or emulsion, or they may be covalently conjugated. The preferred PUFA solution and ratios of such a product (a combination of a PUFA and anticancer drug) is at least 5%, preferably at least 25%, and most preferably about 25-75%.

In another aspect, the invention provides pharmaceutical compositions comprising a PUFA, or a PUFA salt; a TNF-α/IFN (this includes α, β and γ forms) and an anti-cancer drug may be separate chemical moieties combined in a solution or emulsion, or they may be covalently conjugated. The TNF-α/IFN and anti-cancer agents may be mixed with the PUFA solution described above, either to form a new solution or to form an emulsion, or they may be chemically conjugated to the PUFAs of the invention via standard chemistries. Preferably the PUFA solution is mixed with such a TNF-α/IFN and anti-cancer drug in a ratio of at least about 1:1:1, or about 10:1:1, or about 1:10:1, or about 1:1:10 or in any combination or ratio (volume/volume/volume or Mol: Mol: Mol). The PUFA solution may be safely administered to a typical patient with glioma or any other tumor in an amount of about 1 mg to 500 mg in a volume of about 0.5 mL to 10 mL/m² or more as the case may be, but the attending physician should consider all relevant medical factors in determining the appropriate dosage for any specific patient. The preferred PUFA solution and ratios of such a product (a combination of a PUFA and TNF-α/IFN) are as disclosed above. Preferably the final concentration of the PUFA/GLA in such a product is at least 5%, preferably at least 25%, and most preferably about 25-75%.

The PUFA solutions of the present invention are preferably administered intra-tumorally (into the parenchyma of substance of the tumor). In the case of a glioma or brain tumor, the PUFA solutions are injected into the tumor bed even before surgery or after debulking surgery of the glioma mass, inserting a catheter into the tumor bed so that injections of the PUFA solution could be performed on a daily basis after the surgical wound has healed and after confirming the position of the tip of the catheter by CT or MRI or any other feasible and reliable radiographic examination. It is also envisaged that in the treatment of glioma, PUFA solution or GLA solution is delivered to the glioma tumor mass by incorporating PUFA.GLA in a biodegradable wafer or membranes for slowly, steady and predictable amounts of PUFA/GLA to be delivered to the tumor mass anywhere from 24 hours to 1 week or even up to 10 days. The delivery of PUFA/GLA is delivered to the glioma mass by inserting the biodegradable membrane/wafer containing PUFA/GLA at the time of debulking surgery. This invention also envisages a situation where in a patient who has an inoperable glioma; PUFA/GLA could be injected into the tumor mass to shrink its size due to apoptosis of the tumor cells such that the previously inoperable tumor mass now becomes operable and amenable to other treatment modalities including further injections of PUFA/GLA or surgery or radiation or chemotherapy.

It is to be noted in this context that PUFAs can bind to albumin and other proteins and hence, if given intravenously may not be available to be taken up by the tumor cells and consequently may not be able to bring about their tumor cell killing action. In view of this, it is desirable that PUFAs including GLA should be delivered to the patients in such a manner that it is easily available to the tumor (tumor cells) and is delivered selectively to the tumor cells. It is highly desirable that that PUFAs including GLA be given intra-tumorally as was experimentally done in the case of human gliomas. Since, PUFAs can potentiate the cell killing effect of anti-cancer drugs, cytokines (also called as lymphokines), it is desirable to administer a combination of PUFAs, anti-cancer drugs, cytokines such as TNF and IFN or other anti-angiogenic agents or a combination thereof with or without a carrier agent as the situation demands. Further studies have also revealed that PUFAs such as GLA, AA, EPA and DHA can prevent or ameliorate the side effects of anti-cancer agents such as gamma-radiation and cis-platinum to the bone marrow cells of mice. Thus, it appears that when

PUFAs and conventional anti-cancer drugs/agents are given together they not only act to potentiate the cytotoxic action of each other on the tumor cells and thus, produce synergistic and/or additive action on their ability to eliminate the tumor cells but it will also lead to elimination, reduction, or amelioration of the side effects of conventional anti-cancer agents. Since, PUFAs are able to potentiate the cytotoxic action(s) of conventional anti-cancer drugs (agents) and cytokines; it is also possible that this will lead to a significant reduction in the doses of these latter agents without compromising the ultimate benefit namely, elimination of tumor cells or the tumor.

Thus, in another aspect, the invention provides pharmaceutical compositions comprising a PUFA (including GLA), a PUFA salt, and a pharmaceutical agent known in the art for the treatment of neoplasias, either in solution, or in an emulsion. The PUFA (including GLA) and other pharmaceutical agent may be separate chemical moieties combined in the solution or emulsion, or they may be covalently conjugated. The preferred pharmaceutical agents as disclosed above. Preferably the final concentration of the PUFA (including GLA) in such a product is at least 5%, preferably at least 15%, and most preferably at least 25%. The product may contain substantially more PUFA (including GLA), up to 100% without any significant side effects.

The following examples illustrate some preferred modes of practicing the present invention, but are not intended to limit the scope of the claimed invention. Alternative materials and methods may be utilized to obtain similar results.

### EXAMPLES

Studies were conducted in 30 human patients with stage 4 glioma (glioblastoma multiforme, anaplastic astrocytoma and other types of brain tumors).

### Example 1

### Preparation of Gamma-Linolenic Acid (Free Acid) Mixture

Pure GLA (free acid form) was obtained from Sigma chemicals, USA Cayman Chemicals, USA or Nu-Chek Prep, USA and was dissolved in 100% ethyl alcohol. The resultant solution was diluted in normal saline or phosphate buffered saline (PBS), pH 7.4) such that the final concentration of ethyl alcohol ranged from 0.01% to 0.001%. The final concentration of GLA in these solutions was approximately 25% to 90%. The GLA solution was mixed with a stabilizing agent (lithium in the form of lithium chloride). The final concentration of the stabilizing agent ranged from 0.01 to 0.001%. The mixture was prepared under strict sterile conditions.

### Example 2

### Mofication of PUFA

The PUFA is modified by covalent conjugation (e.g., amide bond) to an anti-cancer drug, TNF, IFN, angiostatin, endostatin or other anti-angiogenic substances (especially when the anti-angiogenic action is not needed to its fullest extent or only partial anti-angiogenic action is needed) in a molar or volumetric ratio of at least about 1:1:1, about 10:1:1, about 1:10:1, about 1:1:10 or in any combination or ratio. The mixtures are prepared under strict sterile conditions prior to use.

### Example 3

### Administration of Pharmaceutical Compositions to Patients

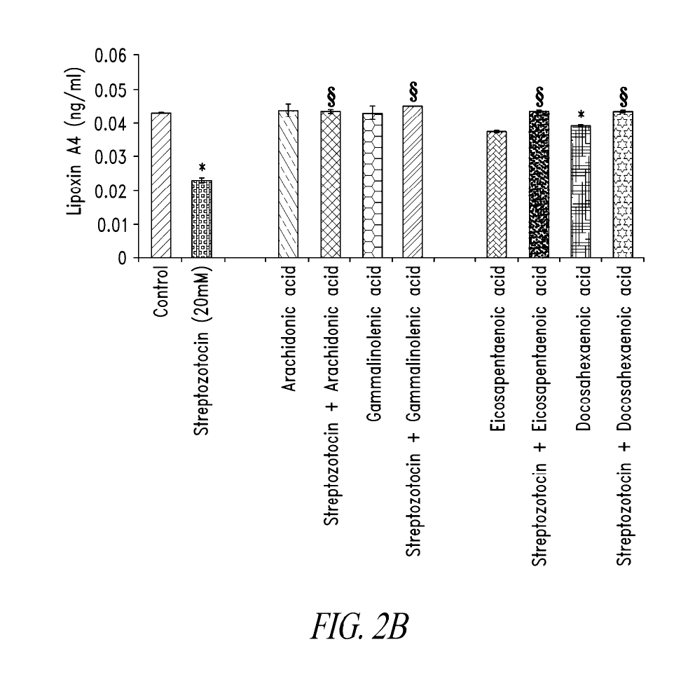

Patients were admitted into the hospital for the study. A complete clinical examination and biochemical assessment of the patient was done including CT/MRI scan of the brain and ultrasound examination of the abdomen to rule out any secondaries in the abdominal organs and an X-ray (radiograph) and/or CT and MRI scan of the chest was performed to assess whether there were any secondaries in the lungs. Carotid angiogram may optionally be performed to deduce the source and extent of blood supply to the glioma (i.e., how many blood vessels are feeding the tumor). CT and Mill scans with contrast medium were performed to evaluate tumor mass, necrosis in the tumor mass and for comparison after treatment. Patients underwent a debulking neurosurgical operation to remove a major portion of the tumor mass. During the surgery, after removing the tumor mass to the extent possible, about 1-10 mg of GLA was instilled into the tumor bed and the wound and bony flap were closed. Before closing the surgical site, a catheter was placed into the tumor bed. The catheter's tip was placed in the tumor bed. The catheter was connected to an Ommaya reservoir (as shown in FIG. 3; or can be connected to a similar subcutaneous pump) and remains beneath the scalp (and can be placed in any other place that is easily accessible for subsequent administration of GLA/PUFA solution). After 7- 10 days following surgery and when the surgical wound has healed well, a CT or MRI scan of the brain was performed to assess the residual glioma or brain tumor remaining after surgery. Then the GLA composition was injected into the glioma tumor bed via the Ommaya reservoir daily for the duration of the therapy (i.e., typically from 7-15 days). Depending on the response of the glioma or brain tumor, the number of doses can be repeated until the tumor regresses to the satisfaction of the treating physician.

### Example 4

### Assessing Tumor Regression Following Treatment with GLA Composition

The degree of regression of the tumor is assessed by performing a CT or MRI scan with or without contrast medium after 7-15 days of administration of the GLA composition and comparing it to the pre-injection picture. If necessary, a repeat carotid angiogram is performed to assess whether blood supply to the glioma or tumor mass has regressed in comparison to the beginning of the study. An angiogram is performed and recorded during and immediately after the injection of the GLA composition, and at periodic intervals thereafter.

### Example 5

### Injection of GLA Acid Composition

30 human patients with stage 4 glioma (glioblastoma multiforme, anaplastic astrocytoma and other types of brain tumors) were treated by direct injection of free acid form of the GLA composition according the examples 1-4.. The first 6 patients with glioma received the free acid form of the GLA composition in doses ranging from 0.25-10 mg of GLA/day for 5-10 days or a total dose of about 4.5 mg to 50 mg of GLA over a period of 7-10 days. All 6 of these patients showed significant reduction in the size of the tumor mass and survived for longer than expected.

### Example 6

### Injection of GLA Free Acid Composition

In another study, 15 patients were injected or infused with the composition comprising the free acid form of GLA in doses varying from 1-10 mg/day for 7 to 10 days, 1 week after the neurosurgical procedure of debulking of the tumor mass. At the time of the surgery, these patients also received instillation of GLA 1-5 mg into the tumor bed at the end of surgery. All patients showed 50-70% reduction in the size of the tumor mass. Reduction was determined by CT and Mill scans performed 1 week after the neurosurgery (before the first injection of GLA after the surgery) compared to CT or MRI scan performed 24 hours after the last injection. Of the 15 patients treated in this fashion, 14 patients survived for more than 2½ to 3 years after the completion of GLA therapy. This is 50-100% longer than expected. Historical data indicates that patients with glioma (especially of glioblastoma multiforme) do not survive for more than 44 weeks (approximately 1 year after the diagnosis of glioma).

### Example 7

### Injection of GLA Free Acid Compostion

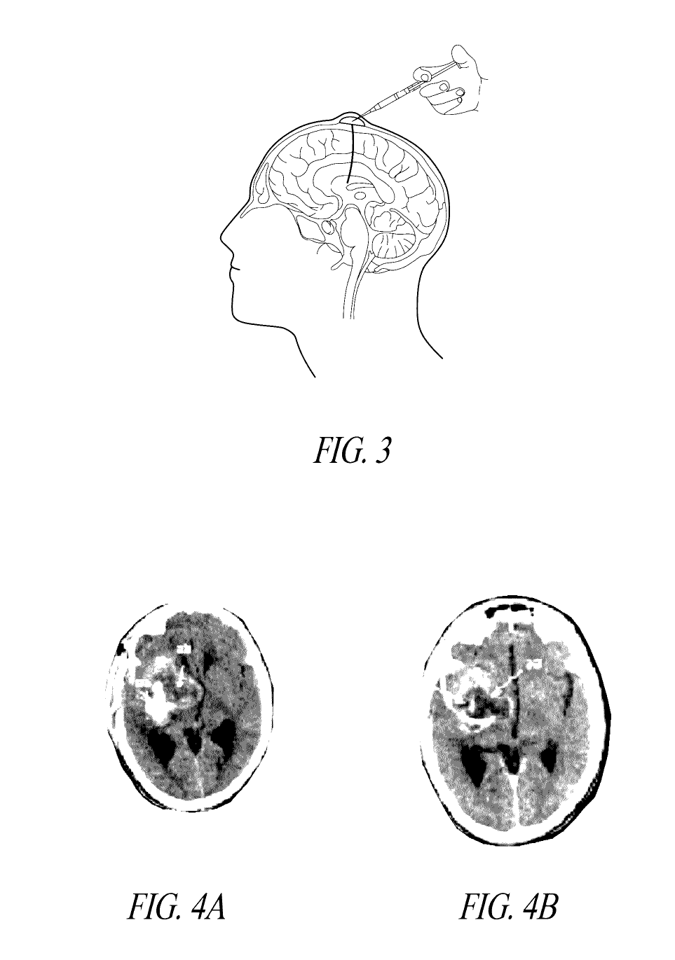

In another study of 9 patients, those who had recurrence of glioma after surgery, radiation and chemotherapy were given the free acid GLA composition at the rate of 1-5 mg GLA/day for 7-10 days also showed regression of tumor mass and improved survival. FIG. 4 shows CT scan of a typical patient who has been treated with GLA recording the regression of tumor mass.

### Example 8

### Effects of PUFAS on Cells In Vitro

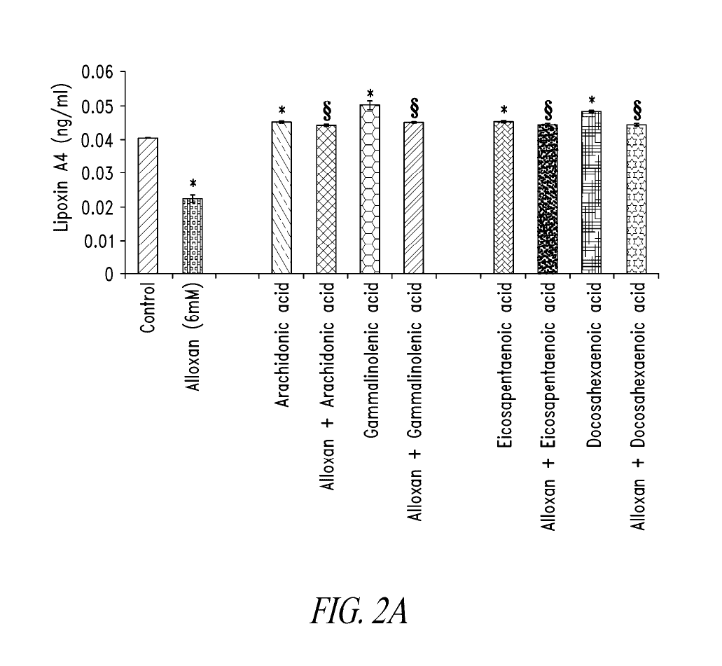

Concentrations of PUFAs used were 15 μg. FIG. 2 illustrates the effect of pretreatment with PUFAs on alloxan and simultaneous treatment with PUFAs and STZ-induced changes in concentrations of LXA4 in RIN cells in vitro. The results show that of all the fatty acids tested, GLA induced generation of LXA4 at a rate greater than or equal to all other fatty acids tested.

### Example 9

### GLA Compositions Treatment Results

A CT scan of the brain of a 15 year old human patient with glioma prior to intratumoral treatment with the disclosed GLA composition is shown in FIG. 4A. The scan shows a left temporal glioblastoma multiforme and marked midline shifts, which suggest raised intracranial tension and mass effect. This is compared to a CT scan taken of the same patient 24 hours after 7 intratumoral injections at the rate of 1 mg/day. The CT scan taken following treatment shows significant necrosis (i.e. black areas within the tumor mass) of the tumor and marked reduction in the midline shift. That suggests reduction in the intracranial tension and mass effect. There were no side effects due to the therapy.

### Example 10

### Effect Ethanol and Stabilizing Agent on GLA Stability and Activity

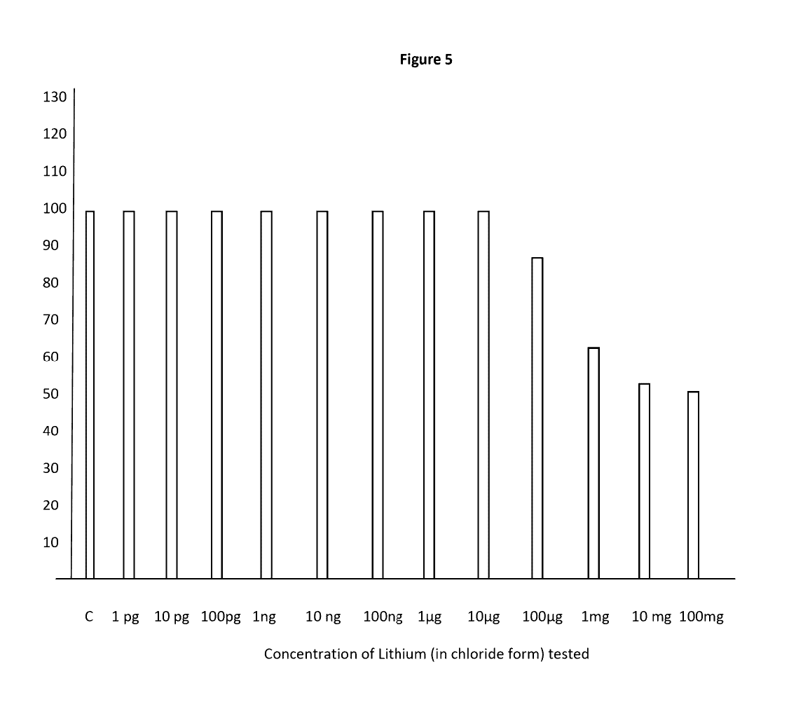

The effect of lithium as an exemplary stabilizing agent in compositions of GLA was tested as follows. To 1 mg (1000 μg) of GLA dissolved in a solution of PBS/saline comprising 0.01% ethanol was added different concentrations of lithium (as lithium chloride), and after a specific period of incubation the concentration of GLA present in the solution was tested. As shown in FIG. 5 (Y axis shows percentage of original GLA remaining, X axis shows mass of lithium per mg of GLA, C is the 100% control), when the ratio between GLA and lithium exceeded about 1:0.01 (GLA: Lithium), the stability of GLA decreased.

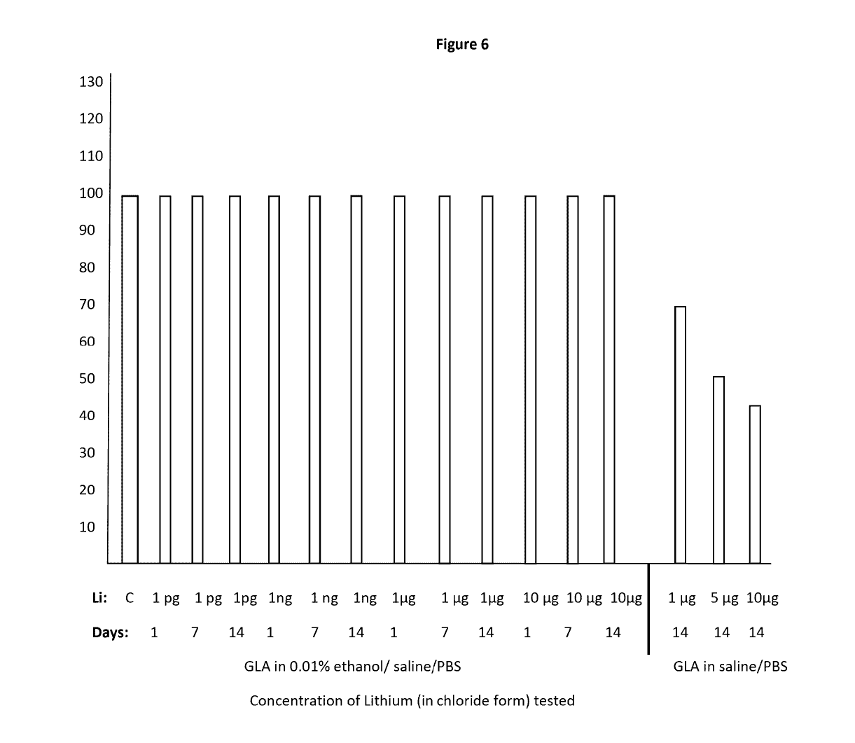

In a separate experiment, GLA was dissolved in saline/PBS (pH 7.4) comprising 0.01% ethanol at 37° C. or saline/PBS (pH 7.4), and different concentrations of lithium was added. At the end of the incubation time the concentration of GLA present was tested. The data in FIG. 6 (Y axis shows percentage of original GLA remaining, X axis shows mass of lithium per mg of GLA at different time points, C is the 100% control) show that ethanol in combination with lithium has a stabilizing effect on GLA, while solutions with ethanol were less stable. The same test system is used to test the effect of Mg, Mn, Zn, Fe, Cu salts on the stability of GLA. The concentrations of these metals used are same as that of Lithium. Relative to lithium, all other metals decrease the concentration of GLA when tested for the same periods of incubation.

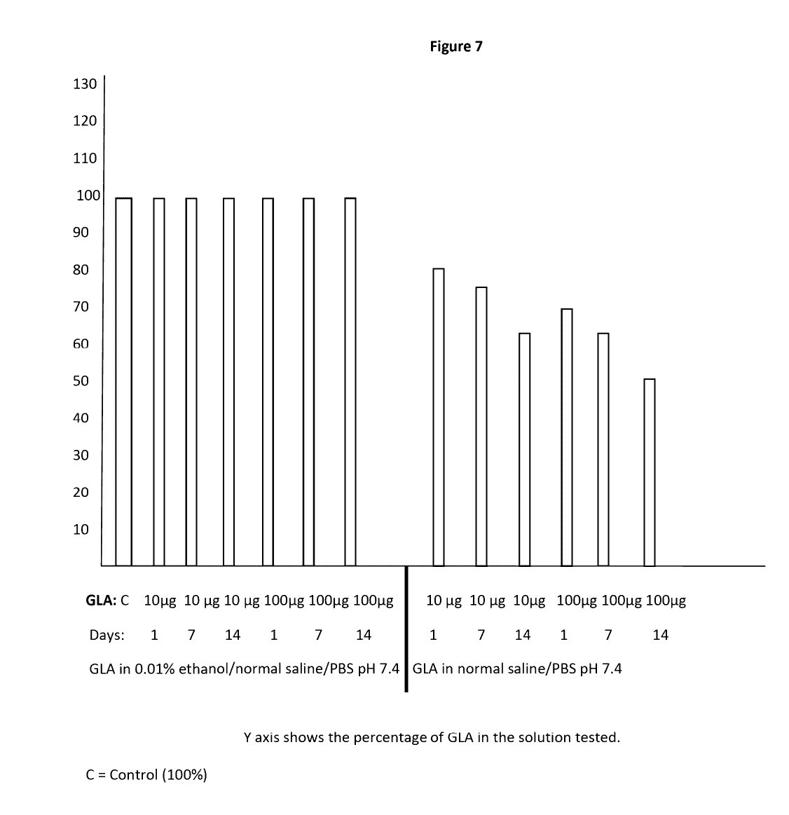

FIG. 7 (Y axis shows percentage of original GLA remaining, C is the 100% control) provides data for GLA dissolved in 0.01% ethanol/saline/PBS (pH 7.4) at 37° C. or saline/PBS (pH 7.4) without added lithium. Again, the data show that ethanol has a stabilizing effect on GLA.

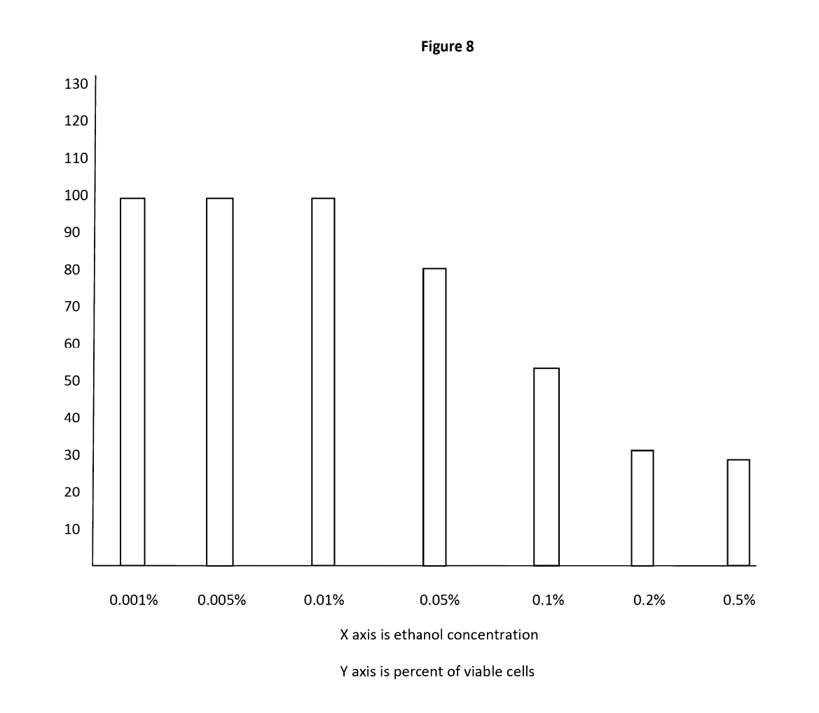

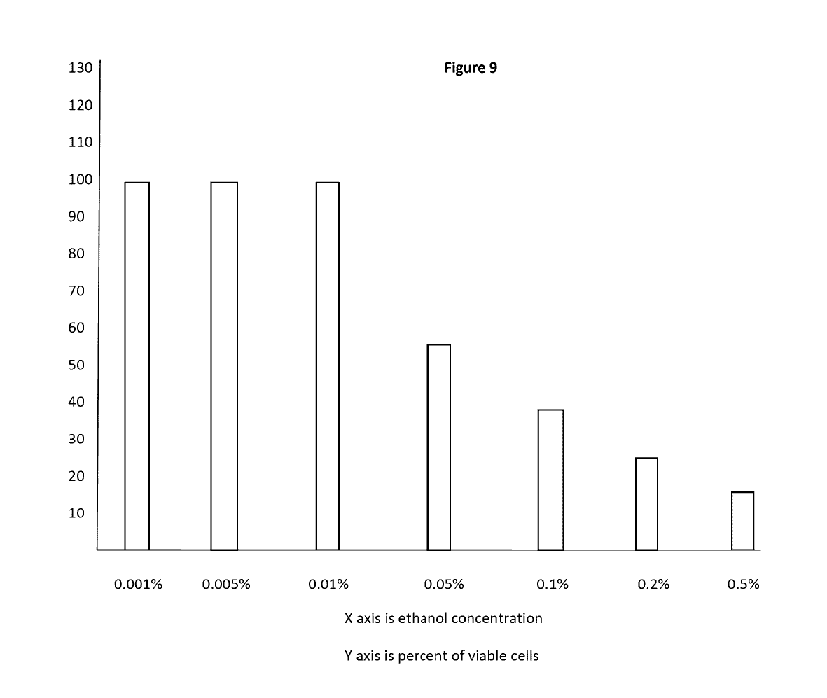

To determine the possible cytotoxic effect of ethanol, compositions comprising 10 μg/ml of GLA in saline/PBS with different concentrations of ethanol were prepared. 1×10⁴ cancer cells or normal cells were incubated with the GLA composition for 72 hrs and viability of the cells in vitro determined. FIG. 8 provides data for cancer cells, and FIG. 9 provides data for normal cells, where the X axis is ethanol concentration, and Y axis is % of viable cells. 10 μg/ml of GLA is believed to be a sub-optimal dose of GLA (i.e., background death of cells is no more than ˜10-15%), therefore any cell death was believed to be associated with ethanol. The data in FIGS. 8 and 9 show that concentrations of ethanol above about 0.01% result in cell death.

The above data provides evidence that ethanol stabilizes GLA in PBS/saline solutions; higher concentrations of ethanol are cytotoxic; and the amount of lithium in the compositions can affect the stability of GLA.

The various embodiments described above can be combined to provide further embodiments. U.S. Provisional Application 62/230,307, filed Jun. 3, 2015, is incorporated herein by reference, in its entirety. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A pharmaceutical composition comprising:
a polyunsaturated fatty acid in the free acid form;
saline or phosphate buffered saline; and
from 0.01% to 0.0001% ethanol.

2. The pharmaceutical composition of claim 1, wherein the polyunsaturated fat is linoleic acid, gamma-linolenic acid, di-homo gamma linolenic acid, arachidonic acid, alpha-linolenic acid, eicosapentaenoic acid, docosahexaenoic acid, conjugated linoleic acid, or combinations thereof.

3. The pharmaceutical composition of claim 1, wherein the polyunsaturated fatty acid is gamma-linolenic acid.

4. The pharmaceutical composition of claim 1, wherein the polyunsaturated fatty acid is conjugated to a tumor necrosis factor a, an interferon, an anti-neoplastic agent, an antibody, an anti-cancer agent or combinations thereof

5. The pharmaceutical composition of claim 4, wherein the anti-cancer agent is vincristine, adriamycin, doxorubicin, cyclophosphamide, cis-platinum, L-asparaginase, procarbazine, camptothecin, taxol, 5-fluorouracil, busulfan, or combinations thereof.

6. The pharmaceutical composition of claim 1, further comprising a tumor necrosis factor a, an interferon, an anti-neoplastic agent, an antibody, an anti-cancer agent or combinations thereof.

7. The pharmaceutical composition of claim 6, wherein the anti-cancer agent is vincristine, adriamycin, doxorubicin, cyclophosphamide, cis-platinum, L-asparaginase, procarbazine, camptothecin, taxol, 5-fluorouracil, busulfan, or combinations thereof.

8. The pharmaceutical composition of claim 4, wherein molar ratio of polyunsaturated fatty acid to tumor necrosis factor a or interferon ranges from 2:1 to 1:3.

9. The pharmaceutical composition of claim 1, wherein the concentration of polyunsaturated fatty acid ranges from 5 to 75%.

10. The pharmaceutical composition of claim 9, wherein the concentration of polyunsaturated fatty acid ranges from 25 to 75%.

11. The pharmaceutical composition of claim 1, further comprising a stabilizing agent.

12. The pharmaceutical composition of claim 11, wherein the stabilizing agent comprises lithium.

13. A method for treatment of cancer comprising:
identifying a neoplastic region in a subject in need thereof;
administering a therapeutically effective amount of the pharmaceutical composition of claim 1 to the neoplastic region.

14. The method of claim 13, wherein administering comprises administering the pharmaceutical composition intratumorally.

15. The method of claim 13, wherein administering comprises administering the pharmaceutical composition via a catheter placed in a tumor bed.

16. The method of claim 13, comprising administering the pharmaceutical composition daily.

17. The method of claim 13, wherein the neoplastic region comprises glioma.

18. The method of claim 13, wherein the composition is steadily and predictably administered in therapeutically effective amounts over 1-10 days.

19. The method of claim 13, wherein the pharmaceutical composition comprises gamma-linolenic acid.

20. A method for preparing a pharmaceutical composition of claim 1, the method comprising,
dissolving a polyunsaturated fatty acid in free acid form in a solution comprising ethanol to form a first mixture; and
diluting the first mixture in saline or phosphate buffered saline such that the final concentration of ethanol ranges from 0.001% to 0.01%.

21. The method of claim 20, wherein the polyunsaturated fatty acid is gamma-linolenic acid.

22. The method of claim 20, further comprising including a stabilizing agent in the first mixture or in the diluted first mixture.

23. The method of claim 22, wherein the stabilizing agent is lithium.
